(19) United States
(12) Patent Application Publication
(10) Pub. No.: US 2005/0244497 A1
(43) Pub. Date: Nov. 3, 2005

(54) Delayed delivery system for acid-sensitive drugs

(75) Inventor: Vinay K Sharma, Long Valley, NJ (US)

Correspondence Address: SCHWEGMAN, LUNDBERG, WOESSNER & KLUTH, P.A., P.O. BOX 2938, MINNEAPOLIS, MN, 55402-0938, US

(73) Assignee: Wockhardt Limited

(21) Appl. No.: 10/478,761

(22) Filed: Nov. 24, 2003

Related U.S. Application Data
(63) Continuation of application No. PCT/US97/20851, filed on Nov. 5, 1997.

(51) Int. Cl. A61K 9/26

(52) U.S. Cl. 424/470

(57) ABSTRACT

The present invention relates to a delayed release drug delivery system containing omeprazole capable of site-specific delivery and pulsatile (bolus) kinetics for once-a-day dosage comprised of an alkaline core structure sequentially layered with suspensions of omeprazole; a separation barrier; and an enteric barrier. The separation barrier is coated with a pH-dependent enteric membrane, which is relatively insoluble in gastric fluid but rapidly to immediately soluble in intestinal fluid, whereby the drug is released in a pulsatile manner in the proximal segment of the gastrointestinal tract.

### RELATED APPLICATIONS

This application is a continuation under 35 U.S.C. 111(a) of International Application No. PCT/US97/20851 filed Nov. 5, 1997 and published in English as WO 98/19668 on May 14, 1998, which claims priority from U.S. application Ser. No. 08/740,981 filed Nov. 6, 1996 (abandoned), which applications and publication are made a part hereof and incorporated herein by reference.

### BACKGROUND OF THE INVENTION

(1) Field of the Invention

The present invention relates to a drug delivery system and, more particularly, to a delayed release drug delivery system for drugs or biologically active materials (e.g., vitamins, vaccines, antibiotics, antifungal agents, muscle relaxers, mood altering drugs, and the like), and especially omeprazole. The system is capable of site-specific delivery and pulsatile (bolus) kinetics.

(2) Description of Prior Art

Omeprazole provides a powerful inhibitory action against secretion of gastric juices and can be used for treatment of gastric and duodenal ulcers. (Lancet, Nov. 27, 1982, pp. 1223-24). However, omeprazole is susceptible to degradation in acidic, acid reactive and neutral media, i.e., the half-life of degradation at pH 4 is less than six minutes, whereas at pH 7 it is about 14 hours. Human pharmacological studies have found that the rate of release of omeprazole can influence the total extent of absorption of the drug to the general circulation. A fully bioavailable dosage form should release the drug rapidly in the proximal part of the gastrointestinal tract.

U.S. Pat. No. 4,786,505 discloses that the stability of conventional formulae of omeprazole is not satisfactory. The reference describes a dosage form including a core region containing omeprazole mixed with alkaline compounds or an alkaline salt of omeprazole mixed with an alkaline compound, or an alkaline omeprazole salt alone coated with one or more layers. The layer/layers which is/are comprised of an alkaline material separates/separate the core material from the outer layer, which is an enteric coating disposed on a subcoating. The final enteric-coated dosage form is treated to reduce water content in order to obtain stability of the drug during long-term storage.

U.S. Pat. No. 4,853,230 describes a pharmaceutical preparation comprising an alkaline-reacting core containing an acid-labile pharmaceutical substance and a pH-buffering alkaline-reacting compound which provides a pH of 7-12 to the micro-environment of the acid labile compound. A subcoating layer is composed of tablet excipients, film forming compounds, and alkaline buffering compounds. Finally, an enteric coating layer surrounds the subcoating layer, wherein the subcoating layer isolates the alkaline-reacting core from the enteric coating layer such that the stability of the product is enhanced.

Omeprazole, along with many other drugs (e.g., Captopril) or biologically active materials (e.g., beta-lactams), vitamins, vaccines, antibiotics, antifungal agents, muscle relaxers, mood altering drugs, and the like can be acid sensitive. That is, their active properties can decrease significantly in the presence of acids, usually by deterioration of the drug or chemical reaction with the drug, altering its composition. The use of enteric layers, which generally comprise layers with carboxyl groups or other acid groups, can adversely affect the stability of the acid sensitive ingredients, since the acid groups on the polymers or other ingredients used in the enteric coating can directly contribute to the acid sensitizing capability of the environment of the pellets during storage.

### SUMMARY OF THE INVENTION

The present invention provides a delayed release drug delivery system containing an acid sensitive drug which is stable at pH levels above 9.0, or biologically active material which is acid sensitive yet stable at pH levels above 9.0, such as omeprazole, for once-a-day dosage. The present invention provides a dose of an acid sensitive drug which is stable at pH levels above 9.0, or biologically active material which is acid sensitive yet stable at pH levels above 9.0, such as omeprazole comprising an alkaline core structure of high cohesiveness and integrity, capable of withstanding the pressure of attrition during a layering operation. One aspect of the present invention is to take advantage of the alkaline core structure, for optimization of release and stability of acid sensitive drugs such as with omeprazole release. Another aspect of the present invention is to provide a delayed release dosage form of an acid sensitive drug, such as omeprazole which is resistant to dissolution in acid media. Another aspect of the invention is to provide at least one sub-separation layer in the pellets adjacent to a layer containing the active acid sensitive material, the sub-separation layer comprising a water soluble/water dispersible polymer and a pharmaceutically acceptable water soluble buffer which can provide a pH of at least 9.0 (depending upon the stabilization needs of the individual compounds), preferably at least 9.5, more preferably at least 10.0, and most preferably (in the case of omeprazole), at least 10.5 or at least 11.0 (up to 12, for example). The use of the high pH buffer materials assures that even in the presence of moisture which could cause migration of acid within the pellets, the high pH buffer would reduce any effect that migratory acid could have on the system.

A further aspect of the present invention is to provide a delayed release dosage form of an acid sensitive drug such as omeprazole which dissolves rapidly in neutral-to-alkaline media. A still further aspect of the present invention is to provide a delayed release dosage form of omeprazole having good stability during long term storage.

The present invention includes both multicompartment pellets and monocompartment pellets for the release of acid sensitive drugs or biologically active materials. The monocompartment pellets, for example, might include a substrate (e.g., nonpareil), a subseparation layer (e.g., a water-soluble or water dispersible binder or polymer (especially cellulose based polymers), such as hydroxypropyl methyl cellulose, hydroxypropyl cellulose incorporates and mixtures thereof (including with other types of water soluble or water insoluble polymers and binders, which two layers define an alkaline core structure) and a layer of deposited oomeprazole, especially as from suspensions of omeprazole or micronized omeprazole, followed by a semipermeable moisture barrier layer, and finally with an enteric barrier layer. An optional finishing layer may also be provided. The preferred alkaline structure is comprised of a core of a blend of a spheronizing/disintegrating agent such as microcrystalline cellulose and an alkaline material such as magnesium trisilicate or trisodium phosphate, although buffers or salts having the pH range of properties described above may be used.

The alkaline core structure is of high integrity and can withstand pressure of attrition during rotor layering operation and subsequent application of an aqueous suspension of omeprazole. The drug layered alkaline core structure is coated with a non-enteric moisture barrier, and then coated with a pH-dependent, enteric membrane, insoluble in simulated gastric fluid but dissolving immediately in intestinal fluid thereby releasing omeprazole by disintegration in the proximal segment of the gastrointestinal tract in a pulsatile manner.

### BRIEF DESCRIPTION OF THE DRAWINGS

A better understanding of the present invention as well as other objects and advantages thereof will become apparent upon consideration of the detailed disclosure thereof, when taken with the accompanying drawings, wherein

FIG. 1 is the dissolution profile of a delayed delivery system for omeprazole at pH 5.2;

FIG. 2 is the dissolution profile of a delayed delivery system for omeprazole at pH 6.0; and

FIG. 3 is the dissolution profile of a delayed delivery system for omeprazole at pH 7.2.

FIG. 4 is a representation of a tablet dose unit within the scope of the present invention.

FIG. 5 is the drug release profile for a Multi-Compartment enteric delayed release system of the present invention with the omeprazole, using trisodium phopshate as the alkaline component.

### DETAILED DESCRIPTION OF THE INVENTION

The delayed release drug delivery system of the present invention containing omeprazole is comprised of an alkaline core structure; layered omeprazole dispersion in aqueous dispersion of water soluble binder, such as hydroxypropylmethyl cellulose, other cellulose esters, water soluble polymers and the like; a separation barrier, which may be a non-enteric moisture barrier; and a delayed release enteric barrier providing gastro-resistant behavior to deliver omeprazole in the proximal segment (pH 5-6) of the gastrointestinal tract.

Microcrystalline cellulose is the preferred binder as it assists in the absorption of water, which is used to create an alkaline micro-environment for each nonpareil central core by providing a hydroxyl ion concentration gradient. Once contact with water is made, there is migration of hydroxyl ions seeking a lower pH, moving toward omeprazole particles to enhance dissolution and to stabilize the omeprazole during dissolution in the acidic medium. A barrier of low water-solubility polymer or resin, such as ethylcellulose, cellulose acetate, or zein is provided to protect omeprazole-layered particles from subsequent delayed release membrane coats containing carboxylic functionalities during the coating operation.

I. Alkaline Core Structure

Basic alkaline material is selected from the group consisting of salts of strong basic cations and weak acidic anions such as Mg²⁺, Ca²⁺, or Al³⁺ and CO⁻²⁻³, OH⁻, and metal oxides including MgO, CaO, and Al₂O₃, at a preferred pH of 9 or greater; organic buffers including Tris (hydroxymethyl) amino methane; and natural clays including montmorillonite and pharmaceutic necessities including sodium glycerophosphate (pH 9.5) and sodium borate (pH 9.5), ratio of omeprazole to alkaline material being from 1:1 to 1:5, preferably 1:1.5-1:2.5.

The alkaline core structure, in association with a spheronizing/disintegrating agent, is prepared by rotor layering nonpareils (e.g., 30/35 or 25/30 mesh, usually between 20 and 50 mesh) with a powder blend of water softenable polymer such as microcrystalline cellulose and an alkaline agent such as trisodium phosphate or magnesium trisilicate powder. A dispersion of hydroxypropyl cellulose is preferably used as a polymeric binding agent for depositing powder blend on the nonpareils. The powder blend and the binding agent are applied at appropriate powder feed rate, spray rate, air volume, and inlet temperature. Ratio of spheronizing/disintegrating agent is from 2:1 to 1:2, preferably 1:1.5 to 1.5:1.

II. Layered Drug Deposit

The layered omeprazole macroparticulates may be obtained by layering micronized omeprazole dispersion in an aqueous dispersion of water soluble/dispersible organic polymeric binders, especially cellulose-based polymers such as hydroxypropyl methylcellulose (traded as Opadry™ Y-5-7095, a registered trademark of Colorcon) or polysiloxane polymers such as Simethicone Emulsion 30% USP on the alkaline deposit cores using rotor layering equipment.

III. Separation Barrier

The separation barrier is then created over the layered drug deposit. After the drug layering operation, the layer may be formed by any commercial process, such as for example, layering over the drug layer a dispersion of trisodium phopshate and Opadry White YS 22-7719, or a semipermeable barrier composed of water soluble or water dispersible polymers (such as cellulose-based polymers, such as cellulose esters, acetates, other derivatives and copolymers), ethylcellulose (e.g., Surelease®, Colorcon), cellulose acetate or zein.

Application of trisodium phosphate (or other pharmaceutically acceptable buffer with a pH greater than 9.0, etc. (as described below) as a separation barrier may be accomplished by any commercially available process such as suspension layering, powder layering, or compression molding the trisodium phosphate (or other bufferO with a water-soluble binder (e.g., lactose or other pharmaceutically acceptable binder, including other sugars or carbohydrates), preferably when both materials are micronized, or dissolving the buffer, e.g., the trisodium phosphate with a cosolvent (e.g., Carbowax®3350 or 8000).

The barrier thus created inhibits the interaction between dissolved omeprazole (or other acid sensitive drug or biologically active material) and carboxylate ions provided by the delayed release pH enteric barrier. Inhibition of such interaction provides protection to omeprazole from degradation and discoloration.

IV. Delayed Release Enteric Barrier

The delayed release enteric barrier is applied on the non-enteric barrier-coated material by any convenient commercial coating or layering process such as rotor-layering or fluidized bed coating, using an aqueous dispersion of the enteric coating composition, such as, for example, 30% w/w of copolymers of methacrylic acid and ethyl acrylate, plasticized with a nontoxic, pharmaceutically acceptable plasticizer, such as organic plasticizers, and especially triethyl citrate. An antiadherant, such as talc, is used to prevent agglomeration of the membrane-coated beads. As enteric coating materials, cellulose acetate phthalate (CAP), hydroxypropyl methylcellulose phthalate (HPMCP-50), polyvinyl acetate phthalate (PVAP), co-polymerized acrylic polymers such as acrylic acid or methacrylic acid/methacrylic acid methyl esters (L30D-55), or similar compounds may be used, preferably HPMCP-50 and L30D-55.

The enteric coating layer also contains pharmaceutically acceptable plasticizers, such as water-soluble triacetin, triethyl citrate, or propylene glycol. The ratio of polymer to plasticizer may be, for example, between 10:1 to 6:1, preferably being 9:1 to 7:3, and more preferably 8.5:2 to 7.5:2; enteric polymers as selected, based on minimal lag time, followed by pulsatile kinetics; and minimal reactive carboxyl groups. Therefore, the organic-based enteric polymer, HPMCP-50 and water-based enteric polymer, L30D-55, are preferred, with an apparent pKa close to 4.3 (e.g., between 4.1 and 4.5). Insofar as the reactivity of carboxyl groups is concerned, all of these enteric materials produce degradation compounds which impart a tint of purple to the omeprazole-layered alkaline core structure. Therefore, in the present invention, a semipermeable barrier is intentionally applied between the omeprazole layer and the enteric membrane, in order to prevent the degradation of omeprazole.

V. Dissolution Profiles

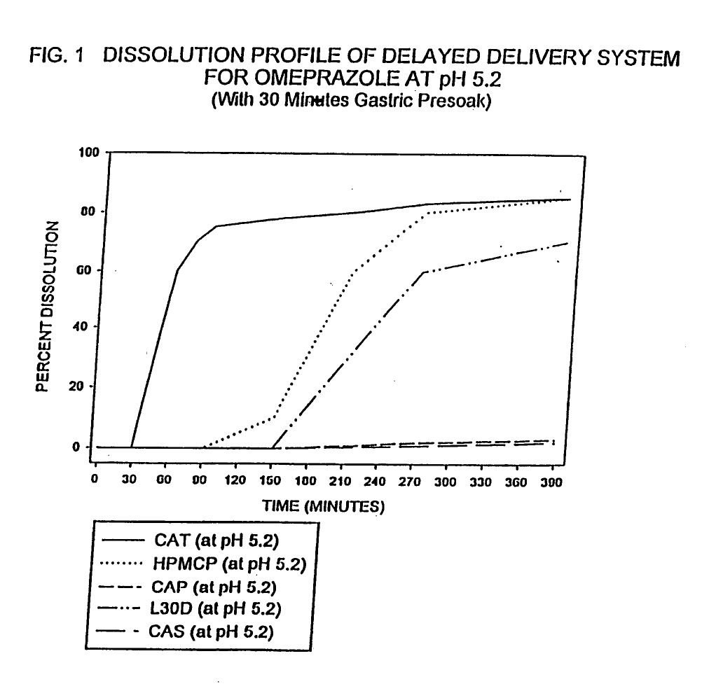

FIG. 1 represents the dissolution profile of a delayed delivery system for omeprazole at pH 5.2, and distinguishes between enteric membranes with regard to lag time and kinetics of drug release. As is evident from FIG. 1, only CAT (cellulose acetate trimellitate) provides an acceptable, pulsatile release kinetics. However, CAT is not acceptable in this instance. Whereas pH 5.2 is acceptable for the fasted, but not for the non-fasted stated, CAT would deliver the drug in the non-fasted stomach where the pH can approach 4-5 in some cases.

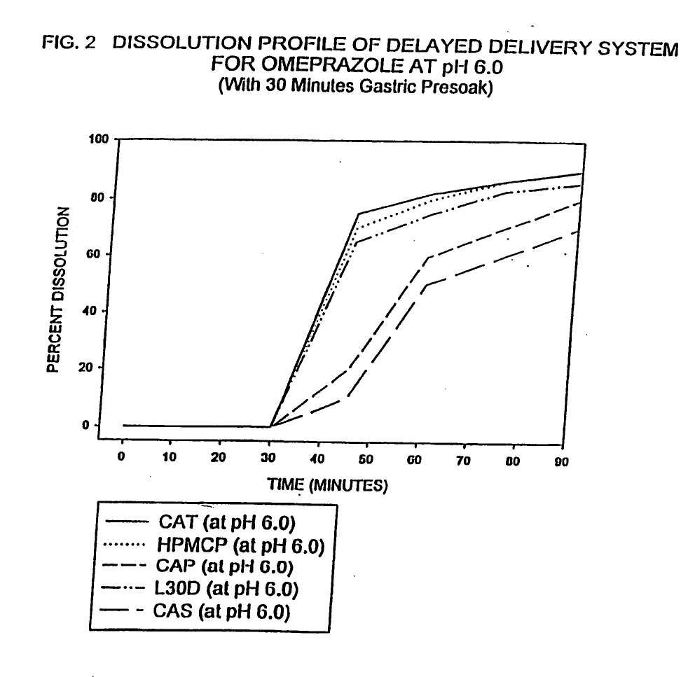

FIG. 2 represents the dissolution profile of a delayed delivery system for omeprazole at pH 6.0, representative of the non-fasted state in the proximal segment of the small intestine. As indicated, HPMCP and L30D-55 provide an optimum pulsatile release profile at pH 6.0. CAT, on the other hand, while providing an optimum pulsatile release profile at pH 6.0, is not acceptable; because of its low pKa, CAT would deliver the drug in the non-fasted stomach. The other materials, CAP and CAS, do not exhibit satisfactory pulsatile release profiles and, in fact, the profiles tend to become sigmoidal in shape.

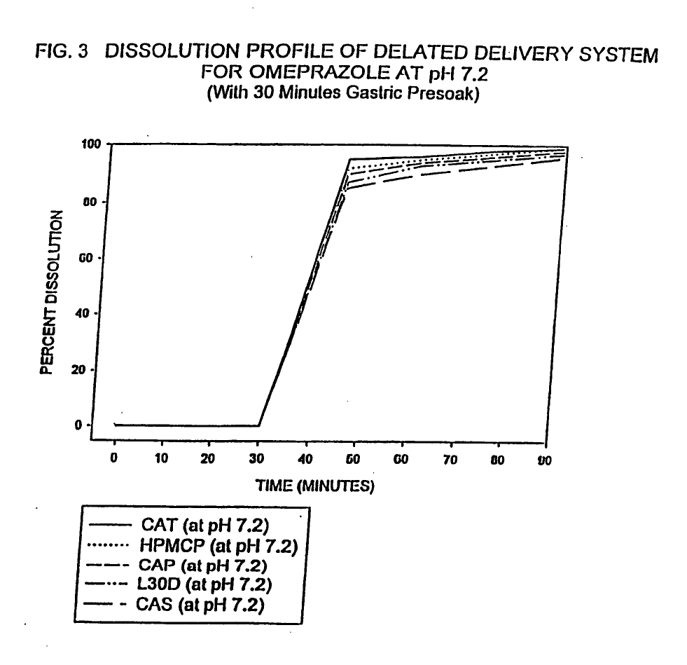

FIG. 3 represents the dissolution profile of a delayed delivery system for omeprazole at pH 7.2. As shown, all enteric barrier materials provide acceptable pulsatile release kinetics; however, a pH of 7.2 approaches that of the large intestine, which is 7.5 to 8.0. Material having a higher pH, 7.2, will not deliver the drug in the proximal segment of the small intestine, where the pH ranges from 4.7 to 6.5. HPMCP and L30D-55 provide most appropriate materials for optimum pulsatile release in the targeted area.

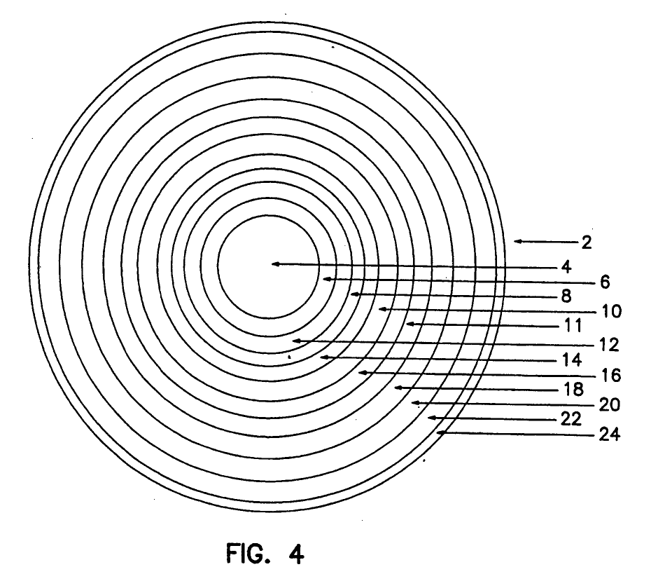

FIG. 4 shows one example of a multicompartment, enteric delayed release system dose unit of the present invention 2 comprising a substrate 4 having alternating subseparation layers 6, 8, 10 and 11, and drug layers 12, 14, 16 and 18. A separation layer 20 overlays all of the previous layers and is in turn covered with an enteric membrane 22 and a finish coat 24.

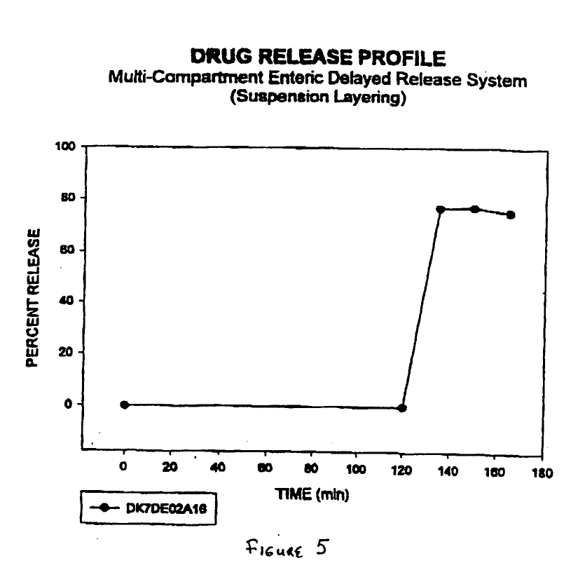

FIG. 5 shows a drug release profile of a multicompartment, enteric delayed release system like that of FIG. 4. As can be seen from the graph, the initial release of the omeprazole was delayed for approximately two hours, with a rapid and steady release occurring quickly over an approximately ten minute period.

### EXAMPLES

The specific nature of the composition of the present invention will be more fully apparent from consideration of the following specific examples of preferred embodiments thereof. In the examples, as in the preceding description, all parts and percentages are given by weight unless otherwise indicated.

### Example 1

### Preparation of Alkaline Core Structure

| | | |
|---|---|---|
| Nonpareils 30/35 mesh | 750 | g. |
| Magnesium Trisilicate | 1500 | g. |
| Microcrystalline Cellulose | 1500 | g. |
| Klucel (6% w/w/ = 4175) | 250.5 | g. |
| Total: | 4000.5 | g. |

The preparation of the alkaline core is achieved by using a blend of a spheronizing agent such as microcrystalline cellulose and an alkaline material such as magnesium trisilicate (preferably, in a 50/50 ratio) which is powder-layered on 30/35 mesh non-pareils, using a 12″ insert in a fluid bed granulator dryer (traded as FLM-15 EX by Vector, Corporation, Marion, Ia.). Particle size analysis is as follows:

| | Mesh | | | | | | |
|---|---|---|---|---|---|---|---|
| | 14 | 16 | 18 | 20 | 25 | 30 | Pan |
| Percent Retained | 2 | 22 | 64 | 10 | 1 | 1 | 4 |

The process yield was 90%. The theoretical potency of magnesium trisilicate is 375%.

### Example 2

### Composition of Suspension for Layered Drug Deo sit

| | | |
|---|---|---|
| Omeprazole | 111 | g. |
| Opadry Y-5-7095 | 44.4 | g. |
| Water, deionized | 954.6 | g. |
| Total: | 1110 | g. |

### Example 3

### Composition of Layered Drug Deposit

| | Solids, g. | %, w/w |
|---|---|---|
| Omeprazole | 111 | 9.607 |
| Opadry Y-5-7095 | 44.4 | 3.843 |
| Alkaline Core Structure | 1000.0 | 86.550 |
| | 1155.4 | 100.000 |

The omeprazole layer dispersion is prepared by weighing purified water into a tared container equipped with a Lightnin Mixer with impeller. With vigorous mixing, hydroxypropyl methylcellulose (Opadry Y-5-7095) is dispersed in water to prepare a smooth paste. Then, remaining water is added to prepare a clear dispersion. To this dispersion, omeprazole (111 g.) is added, to obtain a final omeprazole concentration of 10% w/w. The omeprazole suspension is suspension-layered on magnesium trisilicate pellets, using a 12″ rotor insert in a fluid bed granulator dryer (traded as FLM-15 EX by Vector Corporation, Inc.).

### Example 4

### Preparation of Non-Enteric Moisture Barrier

To the drug deposit core, a 3% weight gain is applied, using a water-based dispersion of ethylcellulose (traded as Surelease® by Colorcon, Inc., Pa.). this is accomplished by using a 12″ rotor insert in the FLM-15 EX fluid bed granulator dryer.

### Example 5

### Delayed Release Enteric Barrier(s)

| | | |
|---|---|---|
| Eudragit L30D-55 | 594.3 | g. |
| Talc, U.S.P. | 35.66 | g. |
| Triethyl Citrate | 35.66 | g. |
| Water, Deionized | 334.33 | g. |
| Total: | 1,000.00 | g. |

A water-based dispersion of Eudragit L30D-55, plasticized with triethyl citrate is applied to provide a weight gain of 15%. An antiadherant talc is used to prevent agglomeration of the membrane-coated beads.

| | | |
|---|---|---|
| Cellulose Acetate Phthalate | 100 | g. |
| Diethyl Phthalate | 25.08 | g. |
| Water, Deionized | 26.25 | g. |
| Acetone, N.F. | 848.75 | g. |
| Total: | 1000.00 | g. |

An organic-based dispersion of cellulose acetate phthalate, plasticized with diethyl phthalate, is applied to provide a weight gain of 15%

| | | |
|---|---|---|
| Cellulose acetate trimellitate, N.F. | 100.08 | g. |
| Triacetin | 25.08 | g. |
| Water, Deionized | 26.25 | g. |
| Acetone, N.F. | 848.75 | g. |
| Total: | 1000.00 | g |

An organic-based dispersion of cellulose acetate trimellitate, plasticized with triacetin, is applied to provide a weight gain of 15%.

| | | |
|---|---|---|
| Hydroxypropyl methylcellulose phthalate | 100.0 | g. |
| (HPMCP-50) | | |
| Acetylated monoglyceride | 15.0 | g. |
| (Myvacet 9-45) | | |
| Water, Deionized | 44.25 | g. |
| Acetone, N.F. | 840.75 | g. |
| | 1000.00 | g. |

An organic-based dispersion of HPMCP-50, plasticized with Myvacet 9-45, is applied to provide a weight gain of 15%. A supercoat of 2% weight gain, using Poadry 7065 (10% w/w dispersion) is used to finish off the coating operation. The potency of omeprazole in imeprazole beads will be as follows:

### Example 6

### Potencies at Various Stages Coating, Percent

| | |
|---|---|
| Drug layered drug deposit = | 9.607 |
| Sub-coated drug layered beads = | 9.444 |
| (2% weight gain based on | |
| alkaline core structure) | |
| Semipermeable barrier coat = | 9.364 |
| (1% weight gain) | |
| Enteric-coated drug layered beads = | 8.312 |
| (15% weight gain) | |
| Super-coated Enteric-coated = | 8.189 |
| drug-layered beads | |
| (2% weight gain) | |

For 20 mg. dosage, therefore, 244 mg. of pellets are required.

### Example 7

Composition of Alkaline Core Structure

| | | |
|---|---|---|
| Nonpareils 25/30 mesh | 750.0 | g |
| Trisodium Phosphate (1.5% w/w) | 31.68 | g |
| Opadry White YS-22-7719 (16.39% w/w) | 21.32 | g |
| TOTAL: | 803.00 | g |

The preparation of the separation layer is achieved by suspension layering on 25/30 mesh nonpareil seeds a dispersion of trisodium phospahe and Opadry White YS-22-7719 in purified water, using a twelve inch insert in a fluidized bed granulator dryer (FLM-15-ES by Vector Corp., Marion, Ia). The total quantity to be applied for five separation layers is 1289.4 grams.

### Example 8

Composition of Drug Layer

| | | |
|---|---|---|
| Omeprazole USP, micronized | 165.0 | g |
| Lactose monohydrate fine powder | 562.9 | g |
| Talc, USP | 29.6 | g |

The powdered drug layer consists of 165.0 grams of micronized Omeprazole USP and 592.5 grams Lactose monohydrate fine powder (micronized) and 29.6 grams of talc. The particle size of omeprazole and the excipients is below 20 microns. The theoretical quantity to be applied to the multilayered separation layered pellets is 757.5 grams.

### Example 9

Theoretical quantity to be applied to the individual separation and rug layers as a total weight of a batch preparation.

| | | |
|---|---|---|
| Sublayer 1 To subsrate | 214.9 | g |
| Drug layer 1 To separation layered pellets | 189.4 | g |
| Sublayer 2 To drug layered pellets | 214.9 | g |
| Drug layer 2 to separation layered pellets | 189.4 | g |
| Sublayer 3 to drug layered pellets | 214.9 | g |
| Drug layer 3 To separation layered pellet | 189.4 | |
| Sublayer 4 to drug layered pellets | 214.9 | g |
| Drug layer 4 To separation layered pellet | 189.4 | |
| Sublayer 5 to drug layered pellets | 429.8 | g |

The total theoretical to be applied to the five separation layers and the four drug layers comprises 1289.4 grams and 757.6 grams, respectively.

### Example 10

Composition of Alkaline Core Structure

| | | |
|---|---|---|
| Nonpareils 25/30 mesh | 750.0 | g |
| Trisodium Phosphate (1.06% w/w) | 5.28 | g |
| Opadry White YS-22-7719 (16.38% w/w) | 35.22 | g |
| TOTAL: | 790.5 | g |

The preparation of the separation layer is achieved by suspension layering on 25/30 mesh nonpareil seeds a dispersion of trisodium phospahe and Opadry White YS-22-7719 in purified water, using a twelve inch insert in a fluidized bed granulator dryer (FLM-15-ES by Vector Corp., Marion, Ia). The total quantity to be applied for the alkaline core structure is 214.9 grams.

### Example 11

Composition of Drug Layer

| | | |
|---|---|---|
| Omeprazole USP, | 135.35 | g |
| Opadry Y-5-7095 | 133.35 | g |
| Simethicone Emulsion 30%, USP | 0.606 | g |
| Total | 267.306 | g |

The suspension of the layered drug deposit is composed of micronized drug particles in a dispersion of Simethicone emulsion 30% USP and Opadry White Y-5-7095 in purified water. Total theoretical quantity to be applied to the four drug layers is 1333.5 grams. The Omeprazole suspension is suspension layered on the alkaline core structure (composed of water-soluble trisodium phosphate), using a twelve inch rotor insert in a fluid bed granulation dryer (FLM-15 ES, Vector Corporation, Marion, Ia).

While the invention has been described in connection with an exemplary embodiment thereof, it will be understood that many modifications will be apparent to those of ordinary skill in the art, and that this application is intended to cover any adaptations of variations thereof. Therefore, it is manifestly intended that this invention be only limited by the claims and the equivalents thereof.

1. A drug delivery system for which comprises a plurality of pellets, each of said pellets comprising a core of a basic alkaline material, a coating of omeprazole surrounding said core of alkaline material; and an enteric membrane, said system being characterized by having
multiple layers of omeprazole separated by non-enteric moisture barriers surrounding omeprazole layers and at least one enteric layer comprising enteric film former plasticized with a water soluble plasticizer, said enteric membranes having a weight gain sufficient to permit release of omeprazole after immersion in both 0.1N HCl for two hours for enteric behavior, followed by pH 6.8 buffer, said release corresponding to a drug release pattern of 0% of the total omeprazole released after at least 1.5 hours of measurement in 0.1N HCl and from 60-80% of the total omeprazole released after 45 minutes of measurement in said pH 6.8 buffer.

2. The drug release system of claim 1 wherein said core comprises both a basic alkaline material and a spheronizing structuring agent.

3. The drug release system of claims 1 and 2 wherein each of said pellets comprise said core and at least three distinct drug layers comprising omeprazole, each of said drug layers being separated from other drug layers by sub-separation layers comprising moisture barrier layers.

4. The drug release system of claims 1 and 2 wherein each of said pellets comprise said core and at least three distinct drug layers comprising omeprazole, each of said drug layers being separated from other drug layers by sub-separation layers comprising moisture barrier layers, and wherein said moisture barrier layers comprise non-enteric moisture barrier comprising water-insoluble semipermeable polymeric membranes.

5. The drug release system of claim 4 wherein said non-enteric moisture barrier layer comprises a cellulose-based resin.

6. The drug release system of claim 3 wherein said at least one enteric layer comprising enteric film former plasticized with a water soluble plasticizer comprises a cellulose-based polymer.

7. The drug release system of claim 3 wherein said enteric coating comprises an acrylic resin.

8. The drug release system of claims 1 and 2 wherein each of said pellets comprise said core and at least three distinct drug layers comprising omeprazole, each of said drug layers being separated from other drug layers by sub-separation layers comprising moisture barrier layers, wherein said at least one enteric layer comprising enteric film former plasticized with a water soluble plasticizer comprises a cellulose-based polymer or acrylic resin, and said water-soluble plasticizer comprises a plasticizer selected from the group consisting of triacetin, triethyl citrate and propylene glycol.

9. A delayed release drug delivery system of omeprazole for site-specific delivery and pulsatile (bolus) kinetics, which comprises a plurality of pellets, each of said pellets having a core of a basic alkaline material and a spheronizing structuring agent; a multi-layer coating of
a) at least one omeprazole layer surrounding said core of alkaline material;
b) a non-enteric moisture barrier surrounding said at least one omeprazole layer; and
c) at least one layer of an enteric membrane comprising enteric film former plasticized with a water soluble plasticizer,
said enteric membrane having a weight gain sufficient to permit release of omeprazole in 0.1N HCl for at least 1.5 hours for enteric behavior, followed by pH 6.8 buffer, corresponding to a drug release pattern of 0% of the total omeprazole released after two hours of measurement in 0.1N HCl and from 60-80% of the total omeprazole released after 45 minutes of measurement in said pH 6.8 buffer.

10. The delayed release drug delivery system as defined in claim 9, wherein said basic alkaline material is selected from the group consisting of salts of strong basic cations and weak acidic anions; organic buffers, natural clays, and sodium borate, the ratio of omeprazole to alkaline material being from 1:1 to 1:5.

11. The delayed release drug delivery system as defined in claim 9, wherein said non-enteric moisture barrier is selected from the group consisting of water-insoluble, semipermeable polymeric membranes of ethylcellulose, cellulose acetate, and zein, wherein said delayed release enteric barrier is selected from the group consisting of acrylic and resin lacquers and being anionic polymers of methacrylic acid, methacrylic acid esters or cellulose phthalic acid ester derivatives, wherein said plasticizer is selected from the group consisting of water-soluble triethyl citrate, triacetin, and propylene glycol, ratio of polymer to plasticizer being 9:1 to 7:3, and wherein the ratio of alkaline material to spheronizing/disintegrating agent is 2:1 to 1:2 in said central alkaline core structure.

12. The delayed release pellets of omeprazole which comprises a plurality of pellets, each of said pellets having a core of a basic alkaline material and a spheronizing structuring agent; a multi-layer coating of omeprazole surrounding said core of alkaline material; a non-enteric moisture barrier surrounding said omeprazole layers; and multilayers of an enteric membrane comprising enteric film form plasticized with a water soluble plasticizer, weight gain of said enteric membranes being sufficient to permit release of omeprazole in 0.1N HCl for two hours for enteric behavior, followed by pH 6.8 buffer, corresponding to a drug release pattern of 0% of the total omeprazole released after two hours of measurement in 0.1N HCl and from 60-80% of the total omeprazole released after 45 minutes of measurement in said pH 6.8 buffer.
